(19) United States
(12) Patent Application Publication
(10) Pub. No.: US 2026/0097090 A1
(43) Pub. Date: Apr. 9, 2026

(54) HERBAL MEDICINE EXTRACTS RM01 AND USES AND METHODS OF PREPARATION THEREOF

(71) Applicant: RenuMemo Therapeutics LLC, Buford, GA (US)

(72) Inventors: Hong YE, Louisville, KY (US); Chengbao YE, Hubei (CN)

(21) Appl. No.: 19/115,183

(22) PCT Filed: Sep. 26, 2023
(86) PCT No.: PCT/US2023/075167

§ 371 Date: Mar. 25, 2025

Related U.S. Application Data
(60) Provisional application No. 63/410,388, filed on Sep. 27, 2022.

(51) Int. Cl. A61K 36/8968; A61K 36/344; A61K 36/815; A61K 36/882; A61P 25/28

(52) U.S. Cl. CPC A61K 36/8968; A61K 36/344; A61K 36/815; A61K 36/882; A61P 25/28

(57) ABSTRACT

The present application provides methods of treating a neurodegenerative disease (such as Alzheimer's disease) in patients by use of a herbal extract composition that has an extract from a mixture including Ophiopogon japonicus, Barbary Wolfberry, Rhizoma Acori tatarinowii and Codonopsis pilosula. Compositions having such extract and the methods of preparing such a composition are also contemplated.

### CROSS-REFERENCE TO RELATED APPLICATION

This application claims priority to U.S. provisional application 63/410,388, filed on Sep. 27, 2022, the content of which is incorporated by reference in its entirety for all purposes.

### FIELD OF THE INVENTION

The present invention relates to the field of herbal medicine and herbal extract composition. In particular, the present invention relates to processed herbal compositions (such as an extract) RM01 comprising Ophiopogon japonicus, Barbary Wolfberry, Rhizoma Acori tatarinowii, and Codonopsis pilosula, and their use in treating a neurodegenerative disease (e.g., Alzheimer's disease) and improving cognition or memory.

### BACKGROUND

In the United States there are currently 6.2 million individuals with Alzheimer's disease (AD). In addition to these with AD in the dementia stages, there are about 10 million individuals with mild cognitive impairment (MCI) (Cummings J et al, Translational Research & Clinical Interventions, 2022). In Alzheimer's disease, the main presenting symptoms are memory loss, difficulty completing familiar tasks, confusion with time or place and challenges in planning or solving problems et al. (Alzheimer's Association). There is no cure for Alzheimer's disease nowadays. A total of 6 drugs were approved by FDA so far, including Aducanumab, which was approved by FDA for the treatment of mild to moderate Alzheimer's disease in 2021. All of the treatments only slow down the Alzheimer's progress but failed to clearly stop progression. The need for therapy to prevent and improve the symptoms of Alzheimer's disease is compelled by the rising number of affected individuals and the growing public health crisis posed by the cost of caring for all the world.

All references described herein are incorporated by reference in their entirety.

### BRIEF SUMMARY OF THE INVENTION

In some embodiments, there is provided a method of treating a neurodegenerative disease or condition in an individual, comprising administering to the individual a herbal extract composition “RM01” comprising an extract of a mixture comprising Ophiopogon japonicus, Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

In some embodiments according to any of the methods described above, the disease or condition is Alzheimer's disease (AD). In some embodiments, the individual has a mild AD or a MMSE score of 20-24. In some embodiments, the individual has a moderate AD or a MMSE score of 13-20. In some embodiments, the individual has a severe AD or a MMSE score that is less than 12.

In some embodiments according to some of the methods described above, the individual has a mild cognitive impairment (MCI) disease.

In some embodiments according to any of the methods described above, the individual has memory loss.

In some embodiments according to any of the methods described above, the herbal extract composition RM01 is orally administered into the individual.

In some embodiments according to any of the methods described above, the individual is a human.

The present application in another aspect provides a herbal extract composition “RM01” comprising an extract of a mixture comprising Ophiopogon japonicus, Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

The present application in another aspect provides a method for preparing an herbal extract composition from a mixture comprising Ophiopogon japonicus, Barbary Wolfberry, Rhizoma Acori tatarinowii and Codonopsis pilosula root, the method comprising: i) adding a liquid to the mixture comprising Ophiopogon japonicus, Barbary Wolfberry, Rhizoma Acori tatarinowii and Codonopsis pilosula to obtain a mixture in the liquid; ii) heating said mixture in the liquid at about the boiling temperature of the liquid, for about 30 minutes to about 60 minutes; iii) collecting a liquid extract from the heated mixture; optionally wherein the liquid is an aqueous solution, further optionally the liquid is water.

The present application in another aspect provides a method for preparing an herbal extract composition from a mixture comprising Ophiopogon japonicus, Barbary Wolfberry, Rhizoma Acori tatarinowii and Codonopsis pilosula, the method comprising: i) adding water to the mixture comprising Ophiopogon japonicus, Barbary Wolfberry, Rhizoma Acori tatarinowii and Codonopsis pilosula to obtain a mixture in water; ii) heating said mixture in water at a temperature of about 100° C., for about 30 minutes to about 60 minutes; iii) collecting a liquid extract from the heated mixture.

In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

The present application in another aspect provides a herbal extract composition prepared by the method described above.

The present application in another aspect provides an herbal extract composition described above, further comprising an excipient.

### BRIEF DESCRIPTION OF THE DRAWINGS

FIG. 1 shows the 2 groups of mice was evaluated by Morris water maze. APP/PS1 are double transgenic mice expressing a chimeric mouse/human amyloid precursor protein (Mo/HuAPP695swe) and a mutant human presenilin 1 (PS1-dE9), both directed to CNS neurons. Both mutations are associated with early-onset Alzheimer's disease. One group was the transgenic mice fed by the herbal extract composition RM01.

FIG. 2 shows the effect of a patient treated by RM01 for 5 weeks.

FIG. 3 shows that RM01 reduced beta amyloid plaques in APP/PS1 mice.

### DETAILED DESCRIPTION OF THE INVENTION

The present invention provides an herbal extraction composition RM01 from a mixture of herbs. The mixture of herbs comprise Ophiopogon japonicus, Barbary Wolfberry, Rhizoma Acori tatarinowii and Codonopsis pilosula. The present invention also provides methods for preparing an herbal extract composition from a mixture of herbs, where the method comprises steps of heating the mixture of herbs in water and collecting the liquid extract formulated in the water. The herbal extract composition RM01 can be used for treating Alzheimer's disease or memory loss in an individual.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms (e.g. Alzheimer's disease), reducing one or more symptoms of a disease, diminishment of extent of disease, preventing or delaying recurrence of disease, delay or slowing of disease progression, amelioration of the disease state, inhibiting the disease or progression of the disease, or arresting its development. Also encompassed by “treatment” is a reduction of pathological consequence of a proliferative disease. The methods of the invention contemplate any one or more of these aspects of treatment. In the context of Alzheimer's disease, the term “treating” includes any or all of: inhibiting the progression of memory loss, improving of overall life quality and ameliorating one or more symptoms associated with the disease, including Alzheimer's disease.

“Therapeutically effective amount” or “effective amount” in the present invention refers to an amount of the herbal extract composition RM01, or the pharmaceutical composition sufficient to improve the condition of the subject in need thereof, without causing serious side-effects.

As used herein, “pharmaceutically acceptable” or “pharmacologically compatible” indicates that a material is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

It is understood that aspects and embodiments of the invention described herein include “comprising,” “consisting,” and “consisting essentially of” aspects and embodiments.

Reference to “about” a value or parameter herein includes (and describes) variations that are directed to that value or parameter per se. For example, description referring to “about X” includes description of “X”.

The term “about” as used herein refers to the usual error range for the respective value readily known to the skilled person in this technical field. Reference to “about” a value or parameter herein includes (and describes) embodiments that are directed to that value or parameter per se.

The term “about X-Y” used herein has the same meaning as “about X to about Y.”

As used herein and in the appended claims, the singular forms “a,” “an,” “or,” and “the” include plural referents unless the context clearly dictates otherwise.

Unless otherwise noted, technical terms are used according to conventional usage.

### Methods of Treatment

The present application provides methods of treating a neurodegenerative disease (e.g., an Alzheimer's disease). Some symptoms of diseases can include, but are not limited to loss of memory, cognition impairment. In one aspect, there is provided a method of treating a disease is a subject in need thereof, wherein the method comprises administering to the individual an herbal extract composition described herein. In some embodiments, the disease is Alzheimer's disease. In some embodiments, the herbal extract composition comprising an extract of a mixture comprising Ophiopogon japonicus, Barbary Wolfberry, Rhizoma Acori tatarinowii and Radix angelicae pubescenti. In some embodiments, the herbal extract composition comprising an extract of a mixture comprising Ophiopogon japonicus, Barbary Wolfberry, Rhizoma Acori tatarinowii and Radix angelicae pubescenti, wherein the herbal extract composition is prepared by a method comprising: i) adding water to the mixture comprising Ophiopogon japonicus, Barbary Wolfberry, Rhizoma Acori tatarinowii and Codonopsis pilosula to obtain a mixture in water; ii) heating said mixture in water at a temperature about 100° C., for about 30 minutes to about 60 minutes; iii) collecting a liquid extract from the heated mixture.

In some embodiments, there is provided a method of treating a neurodegenerative disease (e.g., an Alzheimer's disease) in an individual by administering (e.g., orally administering) a herbal extract composition “RM01” comprising an extract of a mixture comprising at least two or more herbal medicines selected from the group consisting of Ophiopogon japonicus, Barbary Wolfberry, Rhizoma Acori tatarinowii and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising Ophiopogon japonicus and Barbary Wolfberry.

In some embodiments, there is provided a method of treating a neurodegenerative disease (e.g., an Alzheimer's disease) in an individual by administering (e.g., orally administering) a herbal extract composition “RM01” comprising an extract of a mixture comprising Ophiopogon japonicus and Rhizoma Acori tatarinowii.

In some embodiments, there is provided a method of treating a neurodegenerative disease (e.g., an Alzheimer's disease) in an individual by administering (e.g., orally administering) a herbal extract composition “RM01” comprising an extract of a mixture comprising Ophiopogon japonicus and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising Barbary Wolfberry and Rhizoma Acori tatarinowii.

In some embodiments, there is provided a method of treating a neurodegenerative disease (e.g., an Alzheimer's disease) in an individual by administering (e.g., orally administering) a herbal extract composition “RM01” comprising an extract of a mixture comprising Barbary Wolfberry and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising Rhizoma Acori tatarinowii and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising at least three or more herbal medicines selected from the group consisting of Ophiopogon japonicus, Barbary Wolfberry, Rhizoma Acori tatarinowii and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising Ophiopogon japonicus, Barbary Wolfberry, and Rhizoma Acori tatarinowii.

In some embodiments, there is provided a method of treating a neurodegenerative disease (e.g., an Alzheimer's disease) in an individual by administering (e.g., orally administering) a herbal extract composition “RM01” comprising an extract of a mixture comprising Ophiopogon japonicus, Rhizoma Acori tatarinowii and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising Barbary Wolfberry, Rhizoma Acori tatarinowii and Codonopsis pilosula.

In some embodiments, there is provided a method of treating a neurodegenerative disease (e.g., an Alzheimer's disease) in an individual by administering (e.g., orally administering) a herbal extract composition “RM01” comprising an extract of a mixture comprising at least about 10% (w) Ophiopogon japonicus, at least about 10% (w) Barbary Wolfberry, at least about 10% (w) Rhizoma Acori tatarinowii and at least about 10% Codonopsis pilosula. The weight ratio (abbreviated as “w”) refers to the relative weight ratio of the ingredient among the other herb medicines included in the composition. In some embodiments, the other herb medicines consist of Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula. In some embodiments, the individual has a mild AD or a MMSE score of 20-24. In some embodiments, the individual has a moderate AD or a MMSE score of 13-20. In some embodiments, the individual has a severe AD or a MMSE score that is less than 12. In some embodiments, the individual has a mild cognitive impairment (MCI) disease. In some embodiments, the individual has memory loss.

In some embodiments, there is provided a method of treating a neurodegenerative disease (e.g., an Alzheimer's disease) in an individual by administering (e.g., orally administering) a herbal extract composition “RM01” comprising an extract of a mixture comprising Barbary Wolfberry, Rhizoma Acori tatarinowii and Codonopsis pilosula, wherein the weight ratio of Barbary Wolfberry, Rhizoma Acori tatarinowii and Codonopsis pilosula is about 1:1:1:1.

In some embodiments, there is provided a method of improving a symptom associated with a neurodegenerative disease (e.g., Alzheimer's disease) in an individual by administering (e.g., orally administering) a herbal extract composition “RM01” comprising an extract of a mixture comprising Barbary Wolfberry, Rhizoma Acori tatarinowii and Codonopsis pilosula. In some embodiments, the symptom comprises memory loss. In some embodiments, the symptom comprises the deficiency in the individual's ability to follow instructions. In some embodiments, the improvement of a system is indicated by an increase of the MMSE score after treatment. In some embodiments, the individual has demonstrated at least 1, 2, 3, 4, 5, or 6 points of increase in the MMSE score after treatment.

In some embodiments according to any one of the methods described above, the subject is human. In some embodiments, the subject is female. In other embodiments, the subject is male. In some embodiments, the subject is in need of treatment for Alzheimer's disease. In some embodiments, the subject is diagnosed with mild Alzheimer's disease, moderate Alzheimer's disease, or severe Alzheimer's disease.

In some embodiments according to any one of the methods described above, the individual is between about 50 to about 100 years old. In some embodiments, the individual is any one of between about 50 to about 60 years old, between about 60 to about 70 years old, between about 70 to about 80 years old, between about 80 to about 90 years old, or between about 90 to about 100 years old. For memory loss and mild cognitive impairment (MCI), the individual is between about 30 to about 100 years old. In some embodiments, the individual is any one of between about 30 to about 40 years old, between about 40 to about 50 years old, between about 50 to about 60 years old, between about 60 to about 70 years old, between about 70 to about 80 years old, between about 80 to about 90 years old, or between about 90 to about 100 years old.

In some embodiments, the subject has a MMSE score of below 25 prior to the treatment. In some embodiments, the subject has a MMSE score of about 20 to about 24. In some embodiments, the subject has a MMSE score of about any one of 20, 21, 22, 23 or 24. In some embodiments, the subject has a MMSE score of about 13 to about 20. In some embodiments, the subject has a MMSE score of about any one of 13, 14, 15, 16, 17, 18, 19 or 20. In some embodiments, the subject has a MMSE score of about 12 or lower. In some embodiments, the subject has a MMSE score of about any one of 0, 1, 2, 3, 4, 5, 6, 7, 8, 9, 10, 11, or 12.

In some embodiments, the subject has been diagnosed with Alzheimer's disease, wherein one or more symptoms associated with the Alzheimer's disease is not relieved by a prior therapy. In some embodiments, the subject has been diagnosed with Alzheimer's disease, wherein none of the symptoms associated with the Alzheimer's disease is relieved by a prior therapy. Exemplary prior therapies include but are not limited to aducanumab, memantine and/or donepezil.

In one aspect, there is provided a method of ameliorating the symptoms of a neurodegenerative disease in an individual, wherein the method comprises administering to the individual a therapeutically effective amount of an herbal extract composition of according to any one of the embodiments described above. In some embodiments, the disease is Alzheimer's disease. In some embodiments, provided is a method of improving the cognition of an individual suffering from Alzheimer's disease, wherein the method comprises administering to the individual a therapeutically effective amount of an herbal extract composition of according to any one of the embodiments described above.

In some embodiments, the subject receives administration of the herbal extract orally.

In some embodiments according to any one of the methods describe above, the MMSE score of the subject is increased subsequent to administration of the herbal extract. In some embodiments, the MMSE score of the subject receiving administration of the herbal extract is increased as compared to a corresponding individual not receiving administration of the herbal extract. In some embodiments, the MMSE score of the subject receiving administration of the herbal extract is increased as compared to a corresponding individual receiving aducanumab, memantine and/or donepezil. In some embodiments, the MMSE score in at least 50%, 60%, 70%, 80%, or 90% of the subjects is increased subsequent to administration of the herbal extract. In some embodiments, the MMSE score in at least 50%, 60%, 70%, 80%, or 90% of the subjects receiving administration of the herbal extract is increased as compared to a corresponding individuals not receiving administration of the herbal extract. In some embodiments, the MMSE score in at least 50%, 60%, 70%, 80%, or 90% of the subjects receiving administration of the herbal extract is increased as compared to a corresponding individuals receiving aducanumab, memantine and/or donepezil.

In some embodiments according to any one of the methods describe above, wherein the individual has a MMSE score of 0 to about 9 prior to receiving the herbal extract, the MMSE score of an individual is increased by about 1-5, about 5-10, about 10-15, or about 15-20 points after receiving the herbal extract. In some embodiments, the MMSE score of an individual receiving administration of the herbal extract is increased by about 1-5, about 5-10, about 10-15, or about 15-20 points as compared to a corresponding individual not receiving the herbal extract. In some embodiments according to any one of the methods describe above, the MMSE score of an individual receiving administration of the herbal extract is increased by about 1-5, about 5-10, about 10-15, or about 15-20 points as compared to a corresponding individual receiving aducanumab, memantine and/or donepezil. In some embodiments according to any one of the methods describe above, the MMSE score of an individual receiving administration of the herbal extract is increased by about any one of 1, 2, 3, 4, 5, 6, 7, 8, 9, 10, 11, 12, 13, 14 or 15 points as compared to a corresponding individual not receiving the herbal extract. In some embodiments according to any one of the methods describe above, the MMSE score of an individual receiving administration of the herbal extract is increased by 1, 2, 3, 4, 5, 6, 7, 8, 9, 10, 11, 12, 13, 14 or 15 points as compared to a corresponding individual receiving aducanumab, memantine and/or donepezil.

In some embodiments according to any one of the methods describe above, wherein the individual has a MMSE score of about to about 14 prior to receiving the herbal extract, the MMSE score of an individual is increased by about 1-5, about 5-10, or about 10-15 points after receiving the herbal extract. In some embodiments, the MMSE score of an individual receiving administration of the herbal extract is increased by about 1-5, about 5-10, or about 10-15 points as compared to a corresponding individual not receiving the herbal extract. In some embodiments according to any one of the methods describe above, the MMSE score of an individual receiving administration of the herbal extract is increased by about 1-5, about 5-10, or about 10-15 points as compared to a corresponding individual receiving aducanumab, memantine and/or donepezil. In some embodiments according to any one of the methods describe above, the MMSE score of an individual receiving administration of the herbal extract is increased by about any one of 1, 2, 3, 4, 5, 6, 7, 8, 9, 10, 11, 12, 13, 14 or 15 points as compared to a corresponding individual not receiving the herbal extract. In some embodiments according to any one of the methods describe above, the MMSE score of an individual receiving administration of the herbal extract is increased by 1, 2, 3, 4, 5, 6, 7, 8, 9, 10, 11, 12, 13, 14 or 15 points as compared to a corresponding individual receiving aducanumab, memantine and/or donepezil.

In some embodiments according to any one of the methods describe above, wherein the individual has a MMSE score of 15 to about 19 prior to receiving the herbal extract, the MMSE score of an individual is increased by about 1-5, or about 5-10 points after receiving the herbal extract. In some embodiments, the MMSE score of an individual receiving administration of the herbal extract is increased by about 1-5, or about 5-10 points as compared to a corresponding individual not receiving the herbal extract. In some embodiments according to any one of the methods describe above, the MMSE score of an individual receiving administration of the herbal extract is increased by about 1-5, or about 5-10 points as compared to a corresponding individual receiving aducanumab, memantine and/or donepezil. In some embodiments according to any one of the methods describe above, the MMSE score of an individual receiving administration of the herbal extract is increased by about any one of 1, 2, 3, 4, 5, 6, 7, 8, 9, or 10 points as compared to a corresponding individual not receiving the herbal extract. In some embodiments according to any one of the methods describe above, the MMSE score of an individual receiving administration of the herbal extract is increased by 1, 2, 3, 4, 5, 6, 7, 8, 9, or 10 points as compared to a corresponding individual receiving aducanumab, memantine and/or donepezil.

In some embodiments according to any one of the methods describe above, wherein the individual has a MMSE score of 20 to about 24 prior to receiving the herbal extract, the MMSE score of an individual is increased by about 1-5 points after receiving the herbal extract. In some embodiments, the MMSE score of an individual receiving administration of the herbal extract is increased by about 1-5 points as compared to a corresponding individual not receiving the herbal extract. In some embodiments according to any one of the methods describe above, the MMSE score of an individual receiving administration of the herbal extract is increased by about 1-5 points as compared to a corresponding individual receiving aducanumab, memantine and/or donepezil. In some embodiments according to any one of the methods describe above, the MMSE score of an individual receiving administration of the herbal extract is increased by about any one of 1, 2, 3, 4, or 5 points as compared to a corresponding individual not receiving the herbal extract. In some embodiments according to any one of the methods describe above, the MMSE score of an individual receiving administration of the herbal extract is increased by 1, 2, 3, 4, or 5 points as compared to a corresponding individual receiving aducanumab, memantine and/or donepezil.

In some embodiments, MRI and/or PET is used to evaluate the Beta-Amyloid and Tau difference in the individual. In some embodiments, ADAS-Cog13, ADCS-ADL, and/or MoCA is used for cognitive assessment. In some embodiments, the individual has an ADAS-Cog13 change score of less than −4 (e.g., −4, −5, −6, −7, −8, −9, −10).

In some embodiments, the RM01 is administered at least once a day (e.g., once a day or twice a day) for at least about one, two or three days. In some embodiments, the RM01 is administered for at least one, two, three, four, or five weeks. In some embodiments, the RM01 is administered daily or twice a day. In some embodiments, the RM01 is administered once every two days or once every three days. In some embodiments, the RM01 is administered once a week or twice weekly. In some embodiments, the RM01 is administered for at least one month or 2 months. In some embodiments, the RM01 is administered for at least one week, two weeks, three weeks, four weeks, or five weeks.

In some embodiments, the individual is a human patient. In some embodiments, the individual is at least 50 years of age. In some embodiments, the individual has an Alzheimer's disease, optionally wherein the Alzheimer's disease is not relieved by current medication. In some embodiments, the individual has a mild or severe Alzheimer's disease. In some embodiments, the individual has a patient life expectancy greater than 12 months.

The present application further provides methods of reducing or removing amyloid plaques in a brain of an individual comprising administering to the individual a herbal extract composition “RM01” as described above. In some embodiments, the RM01 comprises an extract of a mixture comprising Ophiopogon japonicus, Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula. In some embodiments, the weight ratio of Barbary Wolfberry, Rhizoma Acori tatarinowii and Codonopsis pilosula is about 1:1:1:1. In some embodiments, the RM01 is administered daily or twice a day, optionally wherein the RM01 is administered orally. In some embodiments, RM01 is administered for at least about one, two, three, four or five weeks. In some embodiments, RM01 is administered for at least about one month. In some embodiments, the disease or condition is Alzheimer's disease (AD). In some embodiments, the individual has a mild AD or a MMSE score of 20-24. In some embodiments, the individual has a moderate AD or a MMSE score of 13-20. In some embodiments, the individual has a severe AD or a MMSE score that is less than 12. In some embodiments, the individual has a mild cognitive impairment (MCI) disease. In some embodiments, the individual has memory loss. In some embodiments, the individual is a human.

The present application further provides methods of treating, delaying or preventing a neurodegenerative disease or condition (e.g., Alzheimer's Disease) in an individual comprising administering to the individual a herbal extract composition “RM01” as described above. In some embodiments, the RM01 comprises an extract of a mixture comprising Ophiopogon japonicus, Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula. In some embodiments, the weight ratio of Barbary Wolfberry, Rhizoma Acori tatarinowii and Codonopsis pilosula is about 1:1:1:1. In some embodiments, the RM01 is administered daily or twice a day, optionally wherein the RM01 is administered orally. In some embodiments, RM01 is administered for at least about one, two, three, four or five weeks. In some embodiments, RM01 is administered for at least about one month. In some embodiments, the individual has a mild AD or a MMSE score of 20-24. In some embodiments, the individual has a moderate AD or a MMSE score of 13-20. In some embodiments, the individual has a severe AD or a MMSE score that is less than 12. In some embodiments, the individual has a mild cognitive impairment (MCI) disease. In some embodiments, the individual has memory loss. In some embodiments, the individual is a human.

The present application further provides methods of improving memory of an individual comprising administering to the individual a herbal extract composition “RM01” as described above. In some embodiments, the RM01 comprises an extract of a mixture comprising Ophiopogon japonicus, Barbary Wolfberry, Rhizoma Acori tatarinowii and Codonopsis pilosula. In some embodiments, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula. In some embodiments, the weight ratio of Barbary Wolfberry, Rhizoma Acori tatarinowii and Codonopsis pilosula is about 1:1:1:1. In some embodiments, the RM01 is administered daily or twice a day, optionally wherein the RM01 is administered orally. In some embodiments, RM01 is administered for at least about one, two, three, four or five weeks. In some embodiments, RM01 is administered for at least about one month. In some embodiments, the disease or condition is Alzheimer's disease (AD). In some embodiments, the individual has a mild AD or a MMSE score of 20-24. In some embodiments, the individual has a moderate AD or a MMSE score of 13-20. In some embodiments, the individual has a severe AD or a MMSE score that is less than 12. In some embodiments, the individual has a mild cognitive impairment (MCI) disease. In some embodiments, the individual has memory loss. In some embodiments, the individual is a human.

### Alzheimer's Disease

Alzheimer disease (AD) is biologically defined by the presence of β-amyloid-containing plaques and tau-containing neurofibrillary tangles. AD is a genetic and sporadic neurodegenerative disease that causes an amnestic cognitive impairment in its prototypical presentation and non-amnestic cognitive impairment in its less common variants. AD is a common cause of cognitive impairment acquired in midlife and late-life but its clinical impact is modified by other neurodegenerative and cerebrovascular conditions. This Primer conceives of AD biology as the brain disorder that results from a complex interplay of loss of synaptic homeostasis and dysfunction in the highly interrelated endosomal/lysosomal clearance pathways in which the precursors, aggregated species and post-translationally modified products of Aβ and tau play important roles. Therapeutic endeavours are still struggling to find targets within this framework that substantially change the clinical course in persons with AD. See Knopman et al. Alzheimer disease. Nat Rev Dis Primers 7, 33 (2021).

Ophiopogon japonicus

Ophiopogon japonicus is known as Ophiopogon japonicus (Thunb.) Ker-Gawl, and is also known under the Chinese name of “Mai Dong”. Ophiopogon japonicus is the spindle shade, sharp at the two ends, with length 1.5 to 3 centimeters. It is originally found in Sichuan, Yunnan, Shanxi and Hunan provinces in China. Ophiopogon japonicus is solid, yellowish to white in color and roughly spindle in shape, sharp at the two ends. Ophiopogon japonicus in the raw form has a light pungent smell, and is spicy, bitter and benumbing in taste and not toxic; while in some decocted forms it is light fragrant in smell.

### Barbary Wolfberry

Barbary Wolfberry (also known as Lycium barbarum L.) and is also known under the Chinese name of “Gou Qi.” Barbary Wolfberry is originally found in the northeastern regions in China. In its native form, Barbary Wolfberry is solid, red or light brown in color and is spindle or oval in shape, and is about 6 to 20 mm long and 3 to 10 mm wide. Barbary Wolfberry in the raw form is sweet in taste. It has long history to be used as food supplement.

Rhizoma Acori tatarinowii

Rhizoma Acori tatarinowii is also known under the Chinese name of “Chang Pu.” Three subtypes are typically used, namely Shi Chang Pu (Rhizoma Acori tatarinowii), Jiu Jie Chang Pu (Anemone altaica Fisch. ex C. A. Mey) and Shui Chang Pu (Acorus calamus) and is native to East Asia. In medicine it mainly refers to the dried roots and rhizome of Notopterygium incisum Tncisum Ting ex H. Chang or Notopterygium forbesii Boiss. In the raw form, the root is solid, brown in color and is elongated with circular ridges. In its raw form it has a fragrant smell, and is slightly bitter and pungent in taste. Rhizoma Acori tatarinowii is normally removed from fibrous roots and soil before slicing. In some embodiments, Rhizoma Acori tatarinowii used here refers to its root.

Codonopsis pilosula

Codonopsis pilosula is also known under the Chinese name of “Dang Sheng.” The root is slightly cylindrical, and slightly curved in shape. It is typically about 10 cm to 35 cm in length, 0.4 vm to 2 cm in diameter and is yellowish to grayish. The surface is marked with horizontal wrinkles. It is originally found in Sichuan, Hubei and Anhui provinces in China. In its raw form it is fragrant, and slightly sweet in taste. Codonopsis pilosula is usually used as dry root, and refers to its root in this application.

In some embodiments, the roots of Rhizoma Acori tatarinowii and/or Codonopsis pilosula being used are fresh. In some embodiments, on the roots of Rhizoma Acori tatarinowii and/or Codonopsis pilosula being used are dried.

### Herbal Mixture Composition and Herbal Extract Composition

The present application provides a herbal extract composition RM01 comprising an extract of a mixture comprising Ophiopogon japonicus, Barbary Wolfberry, Rhizoma Acori tatarinowii and Codonopsis pilosula.

In some embodiments, there is provided a herbal extract composition RM01 comprising an extract of a mixture comprising Ophiopogon japonicus, Barbary Wolfberry, Rhizoma Acori tatarinowii and Codonopsis pilosula, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

In one aspect, provided is a composition comprising a mixture (referred to as “herbal mixture” hereafter) comprising Ophiopogon japonicus, Barbary Wolfberry, Rhizoma Acori tatarinowii and Codonopsis pilosula. Through certain extraction methods, an herbal extract composition can be derived from the herbal mixture. Thus in another aspect, provided is an herbal extract composition comprising an extract of a mixture comprising Ophiopogon japonicus, Barbary Wolfberry, Rhizoma Acori tatarinowii and Codonopsis pilosula (the “herbal mixture”).

In some embodiments, the weight percentage of Ophiopogon japonicus in the herbal mixture is about 1-97%. In some embodiments, the weight percentage of Ophiopogon japonicus in the herbal mixture is about 5-85%. In some embodiments, the weight percentage of Ophiopogon japonicus in the herbal mixture is about 10-70%. In some embodiments, the weight percentage of Ophiopogon japonicus in the herbal mixture is about 10-70%. In some embodiments, the percentage by weight of Ophiopogon japonicus in the herbal mixture is any one of: about 1-97%, about 5-85%, about 10-70%, about 15-55%, about 20-40%, or about 20-30%. In some embodiments, the percentage by weight of Ophiopogon japonicus in the herbal mixture is any one of: about 8-15%, about 10-20%, about 60-70%, about 55-70%, or about 55-80%. In some embodiments, the percentage by weight of Ophiopogon japonicus in the herbal mixture is about any one of: about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-97%. In some embodiments, the percentage by weight of Ophiopogon japonicus in the herbal mixture is about any one of: about 1%, about 5%, about 10%, about 12.5%, about 15%, about 20%, about 25%, about 30%, about 35%, about 40%, about 45% about 50%, about 55%, about 60%, about 62.5%, about 65%, about 70%, about 75%, about 80%, about 85%, about 90%, about 95% or about 97%.

In some embodiments, the weight percentage of Barbary Wolfberry in the herbal mixture is about 1-97%. In some embodiments, the weight percentage of Barbary Wolfberry in the herbal mixture is about 5-85%. In some embodiments, the weight percentage of Barbary Wolfberry in the herbal mixture is about 10-70%. In some embodiments, the weight percentage of Barbary Wolfberry in the herbal mixture is about 10-70%. In some embodiments, the percentage by weight of Barbary Wolfberry in the herbal mixture is any one of: about 1-97%, about 5-85%, about 10-70%, about 15-55%, about 20-40%, or about 20-30%. In some embodiments, the percentage by weight of Barbary Wolfberry in the herbal mixture is any one of: about 8-15%, about 10-20%, about 60-70%, about 55-70%, or about 55-80%. In some embodiments, the percentage by weight of Barbary Wolfberry in the herbal mixture is about any one of: about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-97%. In some embodiments, the percentage by weight of Barbary Wolfberry in the herbal mixture is about any one of: about 1%, about 5%, about 10%, about 12.5%, about 15%, about 20%, about 25%, about 30%, about 35%, about 40%, about 45% about 50%, about 55%, about 60%, about 62.5%, about 65%, about 70%, about 75%, about 80%, about 85%, about 90%, about 95% or about 97%.

In some embodiments, the weight percentage of Rhizoma Acori tatarinowii in the herbal mixture is about 1-97%. In some embodiments, the weight percentage of Rhizoma Acori tatarinowii in the herbal mixture is about 5-85%. In some embodiments, the weight percentage of Rhizoma Acori tatarinowii in the herbal mixture is about 10-70%. In some embodiments, the weight percentage of Rhizoma Acori tatarinowii in the herbal mixture is about 10-70%. In some embodiments, the percentage by weight of Rhizoma Acori tatarinowii in the herbal mixture is any one of: about 1-97%, about 5-85%, about 10-70%, about 15-55%, about 20-40%, or about 20-30%. In some embodiments, the percentage by weight of Rhizoma Acori tatarinowii in the herbal mixture is any one of: about 8-15%, about 10-20%, about 60-70%, about 55-70%, or about 55-80%. In some embodiments, the percentage by weight of Rhizoma Acori tatarinowii in the herbal mixture is about any one of: about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-97%. In some embodiments, the percentage by weight of Rhizoma Acori tatarinowii in the herbal mixture is about any one of: about 1%, about 5%, about 10%, about 12.5%, about 15%, about 20%, about 25%, about 30%, about 35%, about 40%, about 45% about 50%, about 55%, about 60%, about 62.5%, about 65%, about 70%, about 75%, about 80%, about 85%, about 90%, about 95% or about 97%.

In some embodiments, the weight percentage of Codonopsis pilosula in the herbal mixture is about 1-97%. In some embodiments, the weight percentage of Codonopsis pilosula in the herbal mixture is about 5-85%. In some embodiments, the weight percentage of Codonopsis pilosula in the herbal mixture is about 10-70%. In some embodiments, the weight percentage of Codonopsis pilosula in the herbal mixture is about 10-70%. In some embodiments, the percentage by weight of Codonopsis pilosula in the herbal mixture is any one of: about 1-97%, about 5-85%, about 10-70%, about 15-55%, about 20-40%, or about 20-30%. In some embodiments, the percentage by weight of Codonopsis pilosula in the herbal mixture is any one of: about 8-15%, about 10-20%, about 60-70%, about 55-70%, or about 55-80%. In some embodiments, the percentage by weight of Codonopsis pilosula in the herbal mixture is about any one of: about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-97%. In some embodiments, the percentage by weight of Codonopsis pilosula in the herbal mixture is about any one of: about 1%, about 5%, about 10%, about 12.5%, about 15%, about 20%, about 25%, about 30%, about 35%, about 40%, about 45% about 50%, about 55%, about 60%, about 62.5%, about 65%, about 70%, about 75%, about 80%, about 85%, about 90%, about 95% or about 97%.

In some embodiments, the herbal mixture comprises Ophiopogon japonicus, Barbary Wolfberry, Rhizoma Acori tatarinowii and Codonopsis pilosula wherein the ratio by weight of Ophiopogon japonicus to Barbary Wolfberry is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Ophiopogon japonicus to Barbary Wolfberry is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Ophiopogon japonicus to Rhizoma Acori tatarinowii is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Ophiopogon japonicus to Rhizoma Acori tatarinowii is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Ophiopogon japonicus to Codonopsis pilosula is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Ophiopogon japonicus to Codonopsis pilosula is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Barbary Wolfberry to Rhizoma Acori tatarinowii is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Barbary Wolfberry to Rhizoma Acori tatarinowii is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Barbary Wolfberry to Codonopsis pilosula is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Barbary Wolfberry to Codonopsis pilosula is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Rhizoma Acori tatarinowii to Codonopsis pilosula is any one of: about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10 or about 1:20. In some embodiments, the ratio by weight of Radix notopterygii to Codonopsis pilosula is about any one of: about 5:1, about 1:1 or about 1:5.

In some embodiments, the herbal mixture comprises Ophiopogon japonicus, Barbary Wolfberry, Rhizoma Acori tatarinowii and Codonopsis pilosula wherein the ratio by weight of Ophiopogon japonicus, Barbary Wolfberry, Rhizoma Acori tatarinowii and Codonopsis pilosula is about any one of: about 1:1:1:2, about 1:1:2:1, about 1:2:1:1, about 2:1:1:1, about 1:1:2:2, about 1:2:1:2, about 1:2:2:1, about 2:1:2:1, about 2:2:1:1, about 2:1:1:2, about 1:2:2:2, about 2:1:2:2, about 2:2:1:2, about 2:2:2:1, about 1:1:1:3, about 1:1:3:1, about 1:3:1:1, about 3:1:1:1, about 1:1:3:3, about 1:3:1:3, about 1:3:3:1, about 3:1:3:1, about 3:3:1:1, about 3:1:1:3, about 1:3:3:3, about 3:1:3:3, about 3:3:1:3, about 3:3:3:1, about 1:1:1:4, about 1:1:4:1, about 1:4:1:1, about 4:1:1:1, about 1:1:4:4, about 1:4:1:4, about 1:4:4:1, about 4:1:4:1, about 4:4:1:1, about 4:1:1:4, about 1:4:4:4, about 4:1:4:4, about 4:4:1:4, about 4:4:4:1, about 1:1:1:5, about 1:1:5:1, about 1:5:1:1, about 5:1:1:1, about 1:1:5:5, about 1:5:1:5, about 1:5:5:1, about 5:1:5:1, about 5:5:1:1, about 5:1:1:5, about 1:5:5:5, about 5:1:5:5, about 5:5:1:5, or about 5:5:5:1.

In one aspect, provided is an herbal extract composition comprising an extract of a mixture comprising Ophiopogon japonicus, Barbary Wolfberry, Rhizoma Acori tatarinowii and Codonopsis pilosula. In some embodiments according to the herbal extract composition described above, the mixture comprises about 1-97% (w) Ophiopogon japonicus, about 1-97% (w) Barbary Wolfberry, about 1-97% (w) Rhizoma Acori tatarinowii and about 1-97% (w) Codonopsis pilosula. In some embodiments according to any one of the herbal extract compositions described above, the mixture comprises about 5-85% (w) Ophiopogon japonicus, about 5-85% (w) Barbary Wolfberry, about 5-85% (w) Rhizoma Acori tatarinowii and about 5-85% (w) Codonopsis pilosula. In some embodiments according to any one of the herbal extract compositions described above, the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula. In some embodiments according to any one of the herbal extract compositions described above, the mixture comprises about 15-55% (w) Ophiopogon japonicus, about 15-55% (w) Barbary Wolfberry, about 15-55% (w) Rhizoma Acori tatarinowii and about 15-55% (w) Codonopsis pilosula. In some embodiments according to any one of the herbal extract compositions described above, the mixture comprises about 20-40% (w) Ophiopogon japonicus, about 20-40% (w) Barbary Wolfberry, about 20-40% (w) Rhizoma Acori tatarinowii and about 20-40% (w) Codonopsis pilosula. In some embodiments according to any one of the herbal extract compositions described above, the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula. In some embodiments according to any one of the herbal extract compositions described above, the mixture comprises about 25% (w) Ophiopogon japonicus, about 25% (w) Barbary Wolfberry, about 25% (w) Rhizoma Acori tatarinowii and about 25% (w) Codonopsis pilosula.

In some embodiments according to any one of the herbal extract compositions described above, the mixture comprises about 55-80% (w) Ophiopogon japonicus, about 8-15% (w) Barbary Wolfberry, about 8-15% (w) Rhizoma Acori tatarinowii and about 8-15% (w) Codonopsis pilosula. In some embodiments according to any one of the herbal extract compositions described above, the mixture comprises about 55-70% (w) Ophiopogon japonicus, about 10-15% (w) Barbary Wolfberry, about 10-15% (w) Rhizoma Acori tatarinowii and about 10-15% (w) Codonopsis pilosula. In some embodiments according to any one of the herbal extract compositions described above, the mixture comprises about 60-70% (w) Ophiopogon japonicus, about 10-20% (w) Barbary Wolfberry, about 10-20% (w) Rhizoma Acori tatarinowii and about 10-20% (w) Codonopsis pilosula. In some embodiments according to any one of the herbal extract compositions described above, the mixture comprises about 60-65% (w) Ophiopogon japonicus, about 10-15% (w) Barbary Wolfberry, about 10-15% (w) Rhizoma Acori tatarinowii and about 10-15% (w) Codonopsis pilosula. In some embodiments according to any one of the herbal extract compositions described above, the mixture comprises about 62.5% (w) Ophiopogon japonicus, about 12.5% (w) Barbary Wolfberry, about 12.5% (w) Rhizoma Acori tatarinowii and about 12.5% (w) Codonopsis pilosula.

In some embodiments according to any one of the herbal extract compositions described above, the mixture comprises about 8-15% (w) Ophiopogon japonicus, about 55-80% (w) Barbary Wolfberry, about 8-15% (w) Rhizoma Acori tatarinowii and about 8-15% (w) Codonopsis pilosula. In some embodiments according to any one of the herbal extract compositions described above, the mixture comprises about 10-15% (w) Ophiopogon japonicus, about 55-70% (w) Barbary Wolfberry, about 10-15% (w) Rhizoma Acori tatarinowii and about 10-15% (w) Codonopsis pilosula. In some embodiments according to any one of the herbal extract compositions described above, the mixture comprises about 10-20% (w) Ophiopogon japonicus, about 60-70% (w) Barbary Wolfberry, about 10-20% (w) Rhizoma Acori tatarinowii and about 10-20% (w) Codonopsis pilosula. In some embodiments according to any one of the herbal extract compositions described above, the mixture comprises about 10-15% (w) Ophiopogon japonicus, about 60-65% (w) Barbary Wolfberry, about 10-15% (w) Rhizoma Acori tatarinowii and about 10-15% (w) Codonopsis pilosula. In some embodiments according to any one of the herbal extract compositions described above, the mixture comprises about 12.5% (w) Ophiopogon japonicus, about 62.5% (w) Barbary Wolfberry, about 12.5% (w) Rhizoma Acori tatarinowii and about 12.5% (w) Codonopsis pilosula.

In some embodiments according to any one of the herbal extract compositions described above, the mixture comprises about 8-15% (w) Ophiopogon japonicus, about 8-15% (w) Barbary Wolfberry, about 55-80% (w) Rhizoma Acori tatarinowii and about 8-15% (w) Codonopsis pilosula. In some embodiments according to any one of the herbal extract compositions described above, the mixture comprises about 10-15% (w) Ophiopogon japonicus, about 10-15% (w) Barbary Wolfberry, about 55-70% (w) Rhizoma Acori tatarinowii and about 10-15% (w) Codonopsis pilosula. In some embodiments according to any one of the herbal extract compositions described above, the mixture comprises about 10-20% (w) Ophiopogon japonicus, about 10-20% (w) Barbary Wolfberry, about 60-70% (w) Rhizoma Acori tatarinowii and about 10-20% (w) Codonopsis pilosula. In some embodiments according to any one of the herbal extract compositions described above, the mixture comprises about 10-15% (w) Ophiopogon japonicus, about 10-15% (w) Barbary Wolfberry, about 60-65% (w) Rhizoma Acori tatarinowii and about 10-15% (w) Codonopsis pilosula. In some embodiments according to any one of the herbal extract compositions described above, the mixture comprises about 12.5% (w) Ophiopogon japonicus, about 12.5% (w) Barbary Wolfberry, about 62.5% (w) Rhizoma Acori tatarinowii and about 12.5% (w) Codonopsis pilosula.

In some embodiments according to any one of the herbal extract compositions described above, the mixture comprises about 8-15% (w) Ophiopogon japonicus, about 8-15% (w) Barbary Wolfberry, about 8-15% (w) Rhizoma Acori tatarinowii and about 55-80% (w) Codonopsis pilosula. In some embodiments according to any one of the herbal extract compositions described above, the mixture comprises about 10-15% (w) Ophiopogon japonicus, about 10-15% (w) Barbary Wolfberry, about 10-15% (w) Rhizoma Acori tatarinowii and about 55-70% (w) Codonopsis pilosula. In some embodiments according to any one of the herbal extract compositions described above, the mixture comprises about 10-20% (w) Ophiopogon japonicus, about 10-20% (w) Barbary Wolfberry, about 10-20% (w) Rhizoma Acori tatarinowii and about 60-70% (w) Codonopsis pilosula. In some embodiments according to any one of the herbal extract compositions described above, the mixture comprises about 10-15% (w) Ophiopogon japonicus, about 10-15% (w) Barbary Wolfberry, about 10-15% (w) Rhizoma Acori tatarinowii and about 60-65% (w) Codonopsis pilosula. In some embodiments according to any one of the herbal extract compositions described above, the mixture comprises about 12.5% (w) Ophiopogon japonicus, about 12.5% (w) Barbary Wolfberry, about 12.5% (w) Rhizoma Acori tatarinowii and about 62.5% (w) Codonopsis pilosula.

In some embodiments according to any one of the herbal extract compositions described above, the mixture comprises about 1-5 kg of Ophiopogon japonicus, about 1-5 kg of Barbary Wolfberry, about 1-5 kg of Rhizoma Acori tatarinowii and about 1-5 kg of Codonopsis pilosula per 6 kg of the mixture. In some embodiments according to any one of the herbal extract compositions described above, the mixture comprises about 1-5 kg of Ophiopogon japonicus, about 1-5 kg of Barbary Wolfberry, about 1-5 kg of Rhizoma Acori tatarinowii and about 1-5 kg of Codonopsis pilosula per 8 kg of the mixture. In some embodiments according to any one of the herbal extract compositions described above, the mixture comprises about 1-5 kg of Ophiopogon japonicus, about 1-5 kg of Barbary Wolfberry, about 1-5 kg of Rhizoma Acori tatarinowii and about 1-5 kg of Codonopsis pilosula per 12 kg of the mixture. In some embodiments according to any one of the herbal extract compositions described above, the mixture comprises about 1-5 kg of Ophiopogon japonicus, about 1-5 kg of Barbary Wolfberry, about 1-5 kg of Rhizoma Acori tatarinowii and about 1-5 kg of Codonopsis pilosula per 16 kg of the mixture. In some embodiments according to any one of the herbal extract compositions described above, the mixture comprises about 1 kg of Ophiopogon japonicus, about 1 kg of Barbary Wolfberry, about 1 kg of Rhizoma Acori tatarinowii and about 1 kg of Codonopsis pilosula per 4 kg of the mixture. In some embodiments according to any one of the herbal extract compositions described above, the mixture comprises about 5 kg of Ophiopogon japonicus, about 1 kg of Barbary Wolfberry, about 1 kg of Rhizoma Acori tatarinowii and about 1 kg of Codonopsis pilosula per 8 kg of the mixture. In some embodiments according to any one of the herbal extract compositions described above, the mixture comprises about 1 kg of Ophiopogon japonicus, about 5 kg of Barbary Wolfberry, about 1 kg of Rhizoma Acori tatarinowii and about 1 kg of Codonopsis pilosula per 8 kg of the mixture. In some embodiments according to any one of the herbal extract compositions described above, the mixture comprises about 1 kg of Ophiopogon japonicus, about 1 kg of Barbary Wolfberry, about 5 kg of Rhizoma Acori tatarinowii and about 1 kg of Codonopsis pilosula per 8 kg of the mixture. In some embodiments according to any one of the herbal extract compositions described above, the mixture comprises about 1 kg of Ophiopogon japonicus, about 1 kg of Barbary Wolfberry, about 1 kg of Rhizoma Acori tatarinowii and about 5 kg of Codonopsis pilosula per 8 kg of the mixture.

### Herbal Extract Composition and Method of Preparation

Certain aspect of the application provides a method for preparing an herbal extract composition from a mixture comprising Ophiopogon japonicus, Barbary Wolfberry, Rhizoma Acori tatarinowii and Codonopsis pilosula, the method comprising: i) adding a liquid to the mixture comprising Ophiopogon japonicus, Barbary Wolfberry, Rhizoma Acori tatarinowii and Codonopsis pilosula; ii) heating said mixture in the liquid at about a boiling temperature for the liquid, for about 30 minutes to about 60 minutes; and iii) collecting a liquid extract from the heated mixture. In some embodiments, the liquid added to the mixture is an aqueous solution. In some embodiments, the liquid added to the mixture is water.

In some embodiments, there is provided a method for preparing an herbal extract composition from a mixture comprising Ophiopogon japonicus, Barbary Wolfberry, Rhizoma Acori tatarinowii and Codonopsis pilosula, the method comprising: i) adding water to the mixture comprising Ophiopogon japonicus, Barbary Wolfberry, Rhizoma Acori tatarinowii and Codonopsis pilosula; ii) heating said mixture in the water at a temperature of about 100 C, for about 30 minutes to about 60 minutes; and iii) collecting a liquid extract from the heated mixture.

In some embodiments, the ratio in weight (w/w) of the liquid (such as water) added versus the volume of the herbal mixture is any one of about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, or about 1:20. In some embodiments, enough liquid (such as water) is added such that herbal mixture is completely submerged in the liquid before extraction. In some embodiments, enough liquid is added such that any solid residue remaining from the herbal mixture is completely submerged in the liquid after extraction. In some embodiments, to each 1 kg of herbal mixture, any one of about 0.1 L, about 0.2 L, about 0.3 L, about 0.4 L, about 0.5 L, about 0.6 L, about 0.7 L, about 0.8 L, about 0.9 L, about 1 L, about 1.5 L, about 2 L, about 2.5 L, about 3 L, about 4 L, about 5 L, about 6 L, about 7 L, about 8 L, about 9 L or about 10 L of liquid is added. In some embodiments, to each 1 kg of herbal mixture, any one of: about 0.1-0.25 L, about 0.25-0.5 L, about 0.5-0.75 L, about 0.75-1 L, about 1-2 L, about 2-3 L, about 3-4 L, about 4-5 L, about 5-6 L, about 6-7 L, about 7-8 L, about 8-9 L, or about 9-10 L of liquid is added.

In some examples, the herbal mixture may be processed before the extraction step. For example, the mixture can be broken down into smaller pieces and/or boiled before heating in the added liquid, to improve extraction efficiency. In some embodiments, the mixture is crushed. In some embodiments, the mixture is ground. In some embodiments, the mixture is cut into smaller pieces. The herbal mixture can be broken down into smaller pieces by any one of: a kitchen knife, pestle and mortar, kitchen blender, fine powder mill grinder or an industrial grinder. In some embodiments, the method comprises grinding the herbal mixture into powder. In some embodiments, the method comprises grinding the herbal mixture into particles with average diameter of about 0.1-0.2 mm, about 0.2-0.3 mm, about 0.3-0.4 mm, about 0.4-0.5 mm, about 0.5-0.6 mm, about 0.6-0.7 mm, about 0.7-0.8 mm, about 0.8-0.9 mm, about 0.9-1.0 mm, about 1-2 mm, 2-5 mm, or about 5-10 mm. In some embodiments, the method comprises grinding the herbal mixture into particles with average diameter of about 5-10 μm, about 10-20 μm, about 20-30 μm, about 30-40 μm, about 40-50 μm, about 50-60 μm, about 60-70 μm, about 70-80 μm, about 80-90 μm, or about 90-100 μm.

The herbal mixture or processed herbal mixture can then be subjected to an extraction step involving heating in a liquid. In some embodiments, the method comprises heating the mixture in the liquid at a temperature of any one of about 40-50° C., about 50-60° C., about 60-70° C., about 70-80° C., about 80-90° C., about 90-100° C., 100-110° C., about 110-120° C., about 120-130° C., about 130-140° C., about 140-150° C., about 150-160° C., about 160-170° C., about 170-180° C., about 180-190° C., or about 190-200° C. In some embodiments, the method comprises heating the mixture in water at a temperature of any one of about 60-80° C., about 80-100° C., or about 100-120° C. In some embodiments, the method comprises heating the mixture in water at a temperature of about 90-100° C. In some embodiments, the method comprises heating the mixture in water at a temperature of about 100° C.

In some embodiments, the method comprises heating the mixture in the liquid at a temperature of no more than about 150° C. In some embodiments, the method comprises heating the mixture in the liquid at temperature no more than any one of about 70° C., about 80° C., about 90° C., about 100° C., about 110° C., about 120° C., about 130° C., about 140° C., or about 150° C. In some embodiments, the method comprises heating the mixture in water at a temperature of no more than about 100° C. In some embodiments, the method comprises heating the mixture in the liquid at temperature no more than any one of about 65° C., about 70° C., about 75° C., about 80° C., about 85° C., about 90° C., about 95° C., or about 100° C.

In some embodiments, the method comprises heating the mixture in the liquid at a temperature of no less than about 50° C. In some embodiments, the method comprises heating the mixture in the liquid at temperature no less than any one of about 40° C., about 45° C., about 50° C., about 55° C., about 60° C., about 65° C., about 70° C., about 75° C., about 80° C., about 85° C., about 90° C., about 95° C., about 100° C., about 110° C., about 120° C., about 130° C., about 140° C. or about 150° C. In some embodiments, the method comprises heating the mixture in water at a temperature of no less than about 50° C. In some embodiments, the method comprises heating the mixture in water at temperature no less than any one of about 40° C., about 45° C., about 50° C., about 55° C., about 60° C., about 65° C., about 70° C., about 75° C., about 80° C., about 85° C., about 90° C., or about 95° C.

The herbal extract may be extracted by heating in the liquid at a temperature slightly below the boiling point of the liquid. Thus in some embodiments, the method comprises heating the mixture in the liquid at a temperature about 1-5° C. or about 5-10° C. below the boiling point of the liquid. In some embodiments, the method comprises heating the mixture in water to about 80-99% of the boiling point temperature of the liquid. For example, in some instances the method comprises heating the mixture in water for about 80-99° C., when the boiling of water is 100° C.

In some embodiments, the boiling point of the liquid is from about 80° C. to about 200° C. In some embodiments, the boiling point of the liquid is any one of about 80-90° C., about 90-100° C., about 100-110° C., about 110-120° C., about 120-130° C., about 130-140° C., about 140-150° C., about 150-160° C., about 160-170° C., about 170-180° C., about 180-190° C., or about 190-200° C.

Absolute pressure is the zero-referenced pressure against perfect vacuum, and is equal to the sum of gauge pressure and the atmospheric pressure, where the atmospheric pressure can vary based on location and altitude. When heating or cooking in a pressure-adjustable device, the absolute pressure is thus the gauge pressure as shown on the device plus the surrounding atmospheric pressure. The absolute pressure under which an herbal medicine mixture is extracted may also affect the active ingredient profiles in the herbal extract composition. Thus, in some embodiments, the method comprises heating the mixture in the liquid at an absolute pressure that is lower than, equal to, or higher than atmospheric pressure at sea level. In some embodiments, the method comprises heating the mixture in a liquid at an absolute pressure of any one of: about 50-60 kPa, about 60-70 kPa, about 70-80 kPa, about 80-90 kPa, about 90-100 kPa, about 100-110 kPa, about 110-120 kPa, about 120-130 kPa, about 130-140 kPa, about 140-150 kPa, about 150-160 kPa, about 160-170 kPa, about 170-180 kPa, about 180-190 kPa, about 190-200 kPa, about 200-210 kPa, about 210-220 kPa, about 220-230 kPa, about 230-240 kPa, about 240-250 kPa, about 250-260 kPa, about 260-270 kPa, about 270-280 kPa, about 280-290 kPa, or about 290-300 kPa, about 300-310 kPa, about 310-320 kPa, about 320-330 kPa, about 330-340 kPa, about 340-350 kPa. For example, the mixture in the liquid (such as water) is heated in a heating device with adjustable pressure, including but not limited to a pressure cooker.

The amount of time the herbs are heated in the liquid can influence the active ingredient profile, and can influence the relative amount of active ingredients extracted. Thus, in some embodiments, the method comprises heating the mixture for about 5 minutes to about 10 hours. In some embodiments, the method comprises heating the mixture for about 5 minutes to about 24 hours. In some embodiments according to any one of the methods described above, the method comprises heating the mixture for about 5-10 minutes, about 10-30 minutes, about 30-60 minutes, about 1-2 hours, about 2-4 hours, about 4-6 hours, about 6-8 hours, about 8-10 hours, about 10-12 hours, about 12-14 hours, about 14-16 hours, about 16-18 hours, about 18-20 hours, about 20-22 hours, or about 22-24 hours. In some embodiments according to any one of the methods described above, the method comprises heating the mixture for about 1-5 minutes, about 5-10 minutes, about 10-20 minutes, about 20-30 minutes, about 30-40 minutes, about 40-50 minutes, about 50-60 minutes, about 1-1.5 hours, about 1.5-2 hours, about 2-3 hours, about 3-4 hours, about 4-5 hours, about 5-6 hours, about 6-7 hours, about 7-8 hours, about 8-9 hours, or about 9-10 hours, about 11-12 hours, about 12-13 hours, about 13-14 hours, about 14-15 hours, about 15-16 hours, about 16-17 hours, about 17-18 hours, about 18-19 hours, about 19-20 hours, about 20-21 hours, about 21-22 hours, about 22-23 hours, or about 23-24 hours. In some embodiments according to any one of the methods described above, the method comprises heating the mixture for any one of: about 2 minutes, about 5 minutes, about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, the method comprises heating the mixture for at least about 30 minutes (such as about any of 0.5, 1, 2, 3, 4, 5, 6, 7, 8, 9, 10 or more hours). In some embodiments, additional liquid can be added to the mixture at one or more time-points during the heating, for example, for one or more times during any one of: about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, or about 23 hours subsequent to the start of heating.

In some embodiments, the liquid extract is collected before the heated mixture is allowed to cool down. In some embodiments, the liquid extract is collected after the heated mixture is allowed to cool down. The liquid extract may be collected by physically excluding the solid residues. In some embodiments, the liquid extract is collected by decanting from the heated mixture. In some embodiments, the liquid extract is collected by passing the heated mixture through one or more constrictions. Many devices contain constrictions suitable for physically excluding the solid residues, including but not limited to sieves, strainers, filters, membranes or matrices of pores. Thus in some embodiments, the liquid extract is collected by passing the heated mixture through one or more sieves, strainers, filters, membranes or matrices of pores. In some embodiments, the diameters of the constrictions through which the liquid extract is passed through are about 5 μm, about 10 μm, about 15 μm, about 20 μm, about 25 μm, about 30 μm, about 40 μm, about 50 μm, about 60 μm, about 70 μm, about 80 μm, about 90 μm, about 100 μm, about 150 μm, about 200 μm, about 300 μm, about 400 μm, about 500 μm, about 600 μm, about 700 μm, about 800 μm, about 900 μm, 1 mm, about 2 mm, about 3 mm, about 4 mm, about 5 mm, or about 10 mm. In some embodiments, the liquid extract is passed through a series of constrictions of decreasing sizes. For example, the heated mixture can be passed through a series of strainers, starting from a 10 mm strainer, through a 1 mm strainer, a 100 μm strainer, and finally a 10 μm strainer, to allow efficient trapping of differently sized residues without clogging the strainers. In some embodiments, the diameter of the constriction used is at least about 1 μm, about 2 μm, about 3 μm, about 4 μm, about 5 μm, about 10 μm, about 20 μm, about 30 μm, about 40 μm, about 50 μm or about 100 μm smaller than the average diameter of the starting herbal mixture. Different extraction parameters such as heating temperature, and duration of heating can affect the profile of active ingredients. After extraction, the liquid extract can comprise the extract of the herbal mixture formulated in the water. Thus in some embodiments, the liquid extract comprises about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-97% water by weight.

In some embodiments, the method further comprises packing and sealing the herbal extract composition RM01 in a sterile container. In some embodiments, the container is a bottle.

Further provided herein are herbal extract compositions prepared by any one of the methods described herein.

For example, in certain aspects, provided is an herbal extract composition comprising an extract of a mixture comprising Ophiopogon japonicus, Barbary Wolfberry, Rhizoma Acori tatarinowii and Radix angelicae pubescenti, wherein the herbal extract composition is prepared by a method comprising: i) adding water to the mixture comprising Ophiopogon japonicus, Barbary Wolfberry, Rhizoma Acori tatarinowii and Codonopsis pilosula to obtain a mixture in water; ii) heating said mixture in water at a temperature about 100° C., for about 30 minutes to about 60 minutes; iii) collecting a liquid extract from the heated mixture. In some embodiments, the herbal extract composition is prepared by any one of the methods described above.

### Pharmaceutical Compositions

The herbal extract composition RM01 described above can be provided in formats applicable for oral administration.

In some embodiments according to any one of the herbal extract described herein, the herbal extract composition is further concentrated. In some embodiments, the herbal extract composition is further concentrated and dried into a powder. In some embodiments, the herbal extract composition is further concentrated such that the concentration is increased by about any one of 10%, 20%, 30%, 40%, 50%, 60%, 70%, 80%, 90%, 100%, 125%, 150%, 175%, 200%, 225%, 250%, 275%, 300%, 350%, 400%, or 500%. In some embodiments, the herbal extract composition is further concentrated such that the concentration is increased by at least about any one of 2-fold, 3-fold, 4-fold, 5-fold, 6-fold, 7-fold, 8-fold, 9-fold, 10-fold, 20-fold, 50-fold. In some embodiments, the herbal extract composition can be air-dried or dried using an incubator, an oven or a desiccator.

In some embodiments, the herbal extract (including but not limited to a further concentrated extract) can be dried as a powder. In some embodiments, the herbal extract can be dried as a powder and formulated in a capsule. In some embodiments, the herbal extract can be dried and formulated as tea bags. In some embodiments, the herbal extract powder can be reconstituted prior to administration (such as with room temperature water, warm water, or hot water). In some embodiments, the herbal extract in a tea bag can be reconstituted by incubating the tea bag in warm water or hot water.

In some embodiments, there is provided a container comprising the herbal extract composition. In some embodiments, the container is opaque. In some embodiments, the container further comprises a label identifying the herbal extract composition. In some embodiments, the container is a bottle. In some embodiments, the container is sterile. Thus in some embodiments, there is provided a sealed bottle comprising the herbal extract composition.

### Method of Treating Diseases

### Kits

Also provided are kits or articles of manufacture for use in treating individuals with a disease, such as Alzheimer's disease. In some embodiments, the kit comprises an herbal extract composition described herein. In some embodiments, the kit comprises an herbal extract composition prepared according to methods described herein. In some embodiments, the kit comprises a composition of herbal mixture described herein. In some embodiments, the kit comprises the compositions described herein (e.g. a composition of herbal mixture described herein, an herbal extract composition described herein, or an herbal extract composition in the form of a patch described herein) in suitable packaging. Suitable packaging materials are known in the art, and include, for example, vials (such as sealed vials), vessels, ampules, bottles, jars, and flexible packaging. These articles of manufacture may further be sterilized and/or sealed. Further provided are the methods of producing the kits or articles of manufacture.

All published references, documents, manuscripts, and scientific literature cited herein are hereby incorporated by reference. While this invention has been particularly shown and described with references to embodiments thereof, it will be understood by those skilled in the art that various changes in form and details may be made therein without departing from the scope of the invention encompassed by the appended claims.

### EXEMPLARY EMBODIMENTS

Embodiment 1. A method of treating a neurodegenerative disease or condition in an individual, comprising administering to the individual a herbal extract composition “RM01” comprising an extract of a mixture comprising Ophiopogon japonicus, Barbary Wolfberry, Rhizoma Acori tatarinowii and Codonopsis pilosula.

Embodiment 2. The method of embodiment 1, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

Embodiment 3. The method of embodiment 1 or embodiment 2, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

Embodiment 4. The method of any one of embodiments 1-3, wherein the disease or condition is Alzheimer's disease (AD).

Embodiment 5. The method of embodiment 4, wherein the individual has a mild AD or a MMSE score of 20-24.

Embodiment 6. The method of embodiment 4, wherein the individual has a moderate AD or a MMSE score of 13-20.

Embodiment 7. The method of embodiment 4, wherein the individual has a severe AD or a MMSE score that is less than 12.

Embodiment 8. The method of any one of embodiments 1-3, wherein the individual has a mild cognitive impairment (MCI) disease.

Embodiment 9. The method of any one of embodiments 1-8, wherein the individual has memory loss.

Embodiment 10. The method of any one of embodiments 1-9, wherein the herbal extract composition RM01 is orally administered into the individual.

Embodiment 11. The method of any one of embodiments 1-10, wherein the individual is a human.

Embodiment 12. A herbal extract composition “RM01” comprising an extract of a mixture comprising Ophiopogon japonicus, Barbary Wolfberry, Rhizoma Acori tatarinowii and Codonopsis pilosula.

Embodiment 13. The herbal extract composition of embodiment 12, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

Embodiment 14. The herbal extract composition of embodiment 12 or 13, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

Embodiment 15. A method for preparing an herbal extract composition from a mixture comprising Ophiopogon japonicus, Barbary Wolfberry, Rhizoma Acori tatarinowii and Codonopsis pilosula root, the method comprising: i) adding a liquid to the mixture comprising Ophiopogon japonicus, Barbary Wolfberry, Rhizoma Acori tatarinowii and Codonopsis pilosula to obtain a mixture in the liquid; ii) heating said mixture in the liquid at about the boiling temperature of the liquid, for about 30 minutes to about 60 minutes; iii) collecting a liquid extract from the heated mixture; optionally wherein the liquid is an aqueous solution, further optionally the liquid is water.

Embodiment 16. A method for preparing an herbal extract composition from a mixture comprising Ophiopogon japonicus, Barbary Wolfberry, Rhizoma Acori tatarinowii and Codonopsis pilosula, the method comprising: i) adding water to the mixture comprising Ophiopogon japonicus, Barbary Wolfberry, Rhizoma Acori tatarinowii and Codonopsis pilosula to obtain a mixture in water; ii) heating said mixture in water at a temperature of about 100° C., for about 30 minutes to about 60 minutes; iii) collecting a liquid extract from the heated mixture.

Embodiment 17. The method of embodiment 15 or embodiment 16, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

Embodiment 18. The method of any one of embodiments 15-17, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

Embodiment 19. A herbal extract composition prepared by the method according to any one of embodiments 15-18.

Embodiment 20. An herbal extract composition of any one of embodiments 12-14 and 19, further comprising an excipient.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1 Preparation of an Herbal Extract Composition

The following steps were performed to prepare the herbal extract composition. The quality of Ophiopogon japonicus, Barbary Wolfberry, Rhizoma Acori tatarinowii roots and Codonopsis pilosula roots were inspected according to quality standards set forth by Chinese Pharmacopoeia. The Rhizoma Acori tatarinowii roots and Codonopsis pilosula roots were then sliced into approximately 1 cm-thick slices. Then, about 1 kg of Ophiopogon japonicus, about 1 kg of Barbary Wolfberry, about 1 kg of Rhizoma Acori tatarinowii root slices and about 1 kg of Codonopsis pilosula root slices were weighed out and boiled in water for 30 minutes to 60 minutes. The extracted liquid part is RM01.

### Example 2 In Vivo Efficacy Determination

To determine the therapeutic efficacy of the herbal extract composition RM01, the herbal extract composition that is being formulated in water was tested in a murine model of APP/PS1.

APP/PS1 mice were used that contain human transgenes for the APP-bearing the Swedish mutation and PSE1 containing an L166P mutation. In these mice, expression of the human APP transgene is approximately 3-fold higher than endogenous murine APP. Amyloid plaque deposition starts at approximately six weeks of age in the neocortex and approximately three to four months in the hippocampus. It is widely used to test the effects of those drugs for learning and memory performance.

RM01 was concentrated by 4 folds from the original concentration. APP/PS1 transgenic mice at 6 months of age received RM01, and evaluated for their performance. Mice were evenly distributed into two groups. Group 1 mice were fed orally with 0.25 ml of concentrated RM01 once per day for two months. Group 2 (the control group) mice were administered by saline. The non-transgenic, wildtype mice were used as the control group 3, which were also fed by saline (n=5-10 mice per group).

The Morris water maze was performed. The apparatus was a circular pool of 120 cm diameter that was filled with water and a platform of 11 cm in diameter was placed for escape. Both groups of mice were brought to the water maze to be trained for 1 to 2 days in the visible platform, and then trained for consecutive 6 to 7 days in the hidden platform. During the hidden platform training, the water in the tank was stained with TiO2, and the platform was placed a depth of 1 cm below the water surface in a constant location, the animals were placed in the random spots of the tank for all trails. On the last day, the platform was removed, and the memory retention functions were evaluated by tracking the animal to probe the platform location in 60 second.

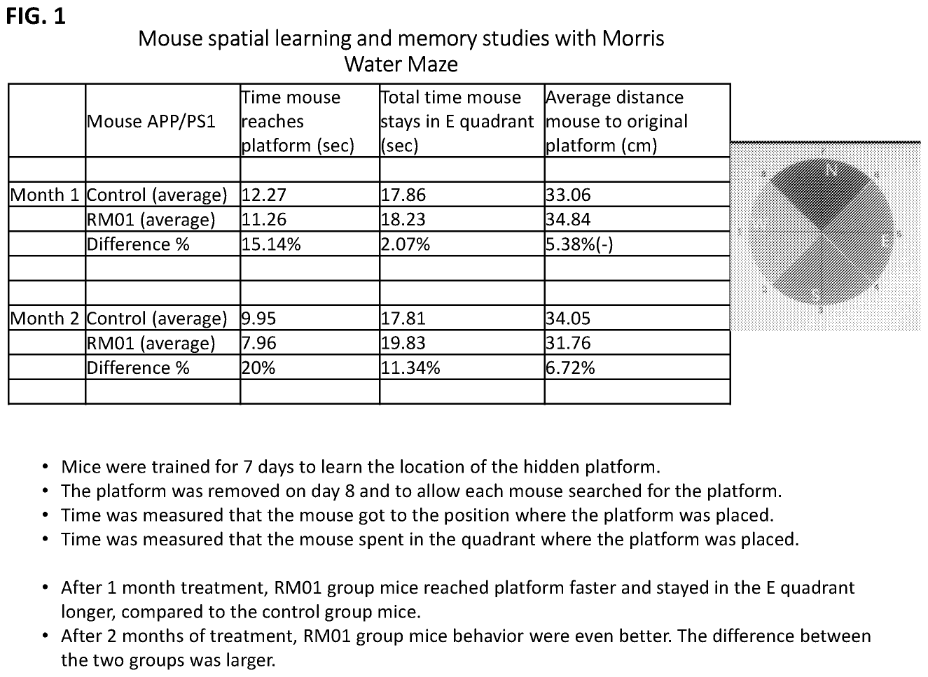

The data was analyzed by Ethovision animal tracking software. The average time of four trials per day that mice spent on searching for the platform during the hidden platform training was plotted a curve to compare the learning potential of mice in each group. On the last day, the memory function of mice was compared by evaluating their performance in probing the platform in the target quadrant, and the detailed parameters include time and length of probing the target quadrant, showing in FIG. 1.

### Example 3 Human Efficacy Studies

A 61-year-old Chinese man, with about 10 year's history since he was first noticed with memory loss affecting daily life. The patient has been taking Memantine and Donepezil to treat his disease. However, both drugs were stopped before the visit. The patient's wife informed that the reason of stopping the medications is not much effect for the patient symptom improvement after long time taking the drugs. Instead, the patient gradually decline in terms of memory, cognition, and daily functions.

The patient was evaluated by Mini Mental State Examination (MMSE) during the first visit. The patient had no sense on time, no instant memory, cannot repeat what the examiner sentence, no sense on number calculation, and cannot follow instructions. The MMSE score was 5, indicated that the patient had severe Alzheimer's disease.

The patient was given the herbal extract composition RM01 twice a day every day, for five weeks. Family member assisted all the process.

The patient was re-evaluated of MMSE after 1 week of treatment. In the meantime, the family member was questioned on patient daily life, regarding sleep, mood, reaction to people, behavioral and psychological symptoms.

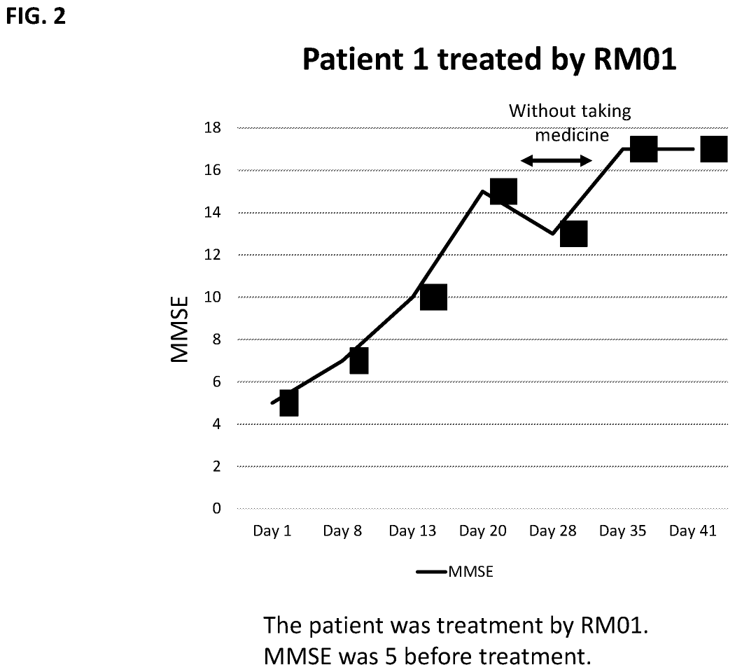

The treatment and re-evaluation lasted 5 weeks. The MMSE score was shown in FIG. 2, suggesting the patient symptoms improved greatly. Family member reported the patient daily life functions increased.

### Example 4

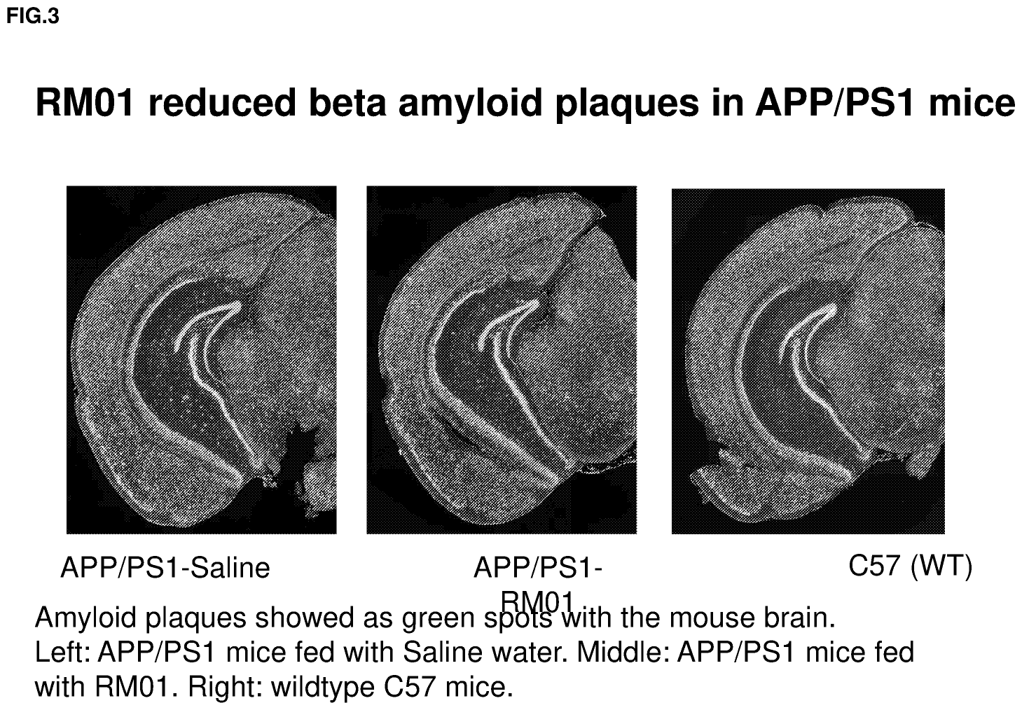

APP/PS1 mice were administered with RM01 to evaluate the RM01 effects on reducing beta amyloid plaques. Each group consisted of four mice, and a total of three groups were assessed: APP/PS1 mice given a saline solution as the control, APP/PS1 mice was given RM01 orally, and C57 wild-type mice also given RM01 as the control. The mice received a daily oral dose of 0.8 ml of RM01, or saline solution for the control group. After two months of treatment, the mice were euthanized and immunohistochemistry testing was conducted against β-amyloid. Two brain slices from each mouse were evaluated, and the total number of β-amyloid was counted and averaged. See FIG. 3 and Table 1.

The control group of C57 wild-type mice exhibited no amyloid plaques, indicating that no artificial plaques were formed due to false data. When compared to the control group of APP/PS1 mice fed with a saline solution, the mice in the APP/PS1 group fed with RM01 showed a significantly reduction in plaques.

TABLE 1

| | Mouse 1 | Mouse 2 | Mouse 3 | Mouse 4 | Average |
|---|---|---|---|---|---|
| APP/PS1- | 53/54 | 60/87 | 77/67 | 91/138 | 78 |
| Saline | | | | | |
| APP/PS1- | 34/50 | 30/27 | 51/62 | 52/41 | 43 |
| RM01 | | | | | |
| C57(WT) | 0/0 | 0/0 | 0/0 | 0/0 | 0 |

What is claimed is:

1. A method of treating a neurodegenerative disease or condition in an individual, comprising administering to the individual a herbal extract composition “RM01” comprising an extract of a mixture comprising Ophiopogon japonicus, Barbary Wolfberry, Rhizoma Acori tatarinowii and Codonopsis pilosula.

2. The method of claim 1, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

3. The method of claim 1 or claim 2, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

4. The method of any one of claims 1-3, wherein the disease or condition is Alzheimer's disease (AD).

5. The method of claim 4, wherein the individual has a mild AD or a MMSE score of 20-24.

6. The method of claim 4, wherein the individual has a moderate AD or a MMSE score of 13-20.

7. The method of claim 4, wherein the individual has a severe AD or a MMSE score that is less than 12.

8. The method of any one of claims 1-3, wherein the individual has a mild cognitive impairment (MCI) disease.

9. The method of any one of claims 1-8, wherein the individual has memory loss.

10. The method of any one of claims 1-9, wherein the herbal extract composition RM01 is orally administered into the individual.

11. The method of any one of claims 1-10, wherein the individual is a human.

12. A herbal extract composition “RM01” comprising an extract of a mixture comprising Ophiopogon japonicus, Barbary Wolfberry, Rhizoma Acori tatarinowii and Codonopsis pilosula.

13. The herbal extract composition of claim 12, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

14. The herbal extract composition of claim 12 or 13, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

15. A method for preparing an herbal extract composition from a mixture comprising Ophiopogon japonicus, Barbary Wolfberry, Rhizoma Acori tatarinowii and Codonopsis pilosula root, the method comprising:
i) adding a liquid to the mixture comprising Ophiopogon japonicus, Barbary Wolfberry, Rhizoma Acori tatarinowii and Codonopsis pilosula to obtain a mixture in the liquid;
ii) heating said mixture in the liquid at about the boiling temperature of the liquid, for about 30 minutes to about 60 minutes;
iii) collecting a liquid extract from the heated mixture;
optionally wherein the liquid is an aqueous solution, further optionally the liquid is water.

16. A method for preparing an herbal extract composition from a mixture comprising Ophiopogon japonicus, Barbary Wolfberry, Rhizoma Acori tatarinowii and Codonopsis pilosula, the method comprising:
i) adding water to the mixture comprising Ophiopogon japonicus, Barbary Wolfberry, Rhizoma Acori tatarinowii and Codonopsis pilosula to obtain a mixture in water;
ii) heating said mixture in water at a temperature of about 100° C., for about 30 minutes to about 60 minutes;
iii) collecting a liquid extract from the heated mixture.

17. The method of claim 15 or claim 16, wherein the mixture comprises about 10%-70% (w) Ophiopogon japonicus, about 10%-70% (w) Barbary Wolfberry, about 10%-70% (w) Rhizoma Acori tatarinowii and about 10%-70% (w) Codonopsis pilosula.

18. The method of any one of claims 15-17, wherein the mixture comprises about 20%-30% (w) Ophiopogon japonicus, about 20%-30% (w) Barbary Wolfberry, about 20%-30% (w) Rhizoma Acori tatarinowii and about 20%-30% (w) Codonopsis pilosula.

19. A herbal extract composition prepared by the method according to any one of claims 15-18.

20. An herbal extract composition of any one of claims 12-14 and 19, further comprising an excipient.
